(19) United States
(12) Patent Application Publication
(10) Pub. No.: US 2009/0187024 A1
(43) Pub. Date: Jul. 23, 2009

(54) ROPIVACAINE HYDROCHLORIDE ANHYDRATE AND THE PREPARATION THEREOF

(75) Inventors: Janmejay Rajnikant Vyas, London (GB); Venkata Satya Varma Nidadavolu, Bopal (IN); Denish Hasmukhlal Shah, Ahmedabad (IN)

Correspondence Address: YOUNG & THOMPSON, 209 Madison Street, Suite 500, ALEXANDRIA, VA, 22314, US

(73) Assignee: DISHMAN PHARMACEUTICALS AND CHEMICALS LTD., AHMEDABAD (IN)

(21) Appl. No.: 12/298,589

(22) PCT Filed: Apr. 25, 2007
(86) PCT No.: PCT/NL2007/050180

§ 371 Date: Jan. 16, 2009

Related U.S. Application Data
(60) Provisional application No. 60/794,519, filed on Apr. 25, 2006.

(30) Foreign Application Priority Data
Apr. 25, 2006 (EP) 06113074.6

(51) Int. Cl. C07D 211/32

(52) U.S. Cl. 546/225

(57) ABSTRACT

The invention pertains to a process for preparing stable anhydrous Ropivacaine hydrochloride, the process involving preparing Ropivacaine hydrochloride from Ropivacaine base, wherein Ropivacaine base is provided having a chiral purity of more than 95%, and wherein the isolation involves the use of isopropanol and hydrochloride, and is performed under water-free conditions. Ropivacaine base may be provided at high chiral purity by N-propylating L-pipecolic acid 2,6-xylidide hydrochloride in the presence of a phase transfer catalyst, wherein the reaction involves a biphasic reaction mixture containing an alkaline aqueous phase and an organic phase. The invention further pertains to stable anhydrous Ropivacaine hydrochloride obtainable by the above process.

### FIELD OF THE INVENTION

The invention pertains to stable Ropivacaine hydrochloride anhydrate and Ropivacaine base having high chiral purity, and the preparation thereof.

### BACKGROUND OF THE INVENTION

WO-A-85/00599 discloses the preparation of (S)-(−)-1-propylpipecolic acid 2,6-xylidide, or Ropivacaine, which compound has proven to be an effective local anesthetic, with unexpectedly long duration compared to the racemate and the corresponding R-(+)-enantiomer, or, for that matter, to Mepivacaine and Bupivacaine, which are its 1-methyl homologue and 1-n-butyl homologue, respectively.

The preparation involves four steps, i.e. a) resolving pipecolic acid using L-(+)-tartaric acid, to isolate the laevo optical stereoisomer; b) chlorination of the stereoisomer, to form L-pipecolic acid chloride hydrochloride; c) reacting the acid chloride hydrochloride with 2,6-xylidine, to form L-pipecolic acid 2,6-xylidide; and d) propylation thereof, to yield the desired Ropivacaine hydrochloride.

The idea of using a resolution method in resolving pipecolic acid in step a) to obtain the longer acting single enantiomers of the local anesthetics Mepivacaine and Bupivacaine was published in J. Med. Chem. (1971) 14:891-892, using tartaric acid monohydrate, whereby isopropanol was added to separate the isopropanol-insoluble enantiomer, whereafter the desired enantiomer was isolated. However, U.S. Pat. No. 5,959,112 teaches that using isopropanol does not give a crystallisation system which is stable during the time required for production in the plant. This is because the solution is supersaturated with the undesired enantiomer, and thus a crystallisation of the undesired enantiomer form could easily be started by small disturbances which means that isopropanol is not suitable to use for production in large scale. The use of isopropanol in combination with various water contents for the resolution steps does not give any improvement.

Similar to WO-A-85/00599, WO-A-96/12700 and EP-A-1.433.782 teach the preparation of racemic and enantiopure Bupivacaine hydrochloride, starting from L-pipecolic acid. Although these publications focus on the beneficial effects of a one-pot synthesis, it involves relatively large amounts of the undesired base enantiomer as an intermediate.

This is emphasised in EP-A-239.710, according to which the preparation method described in WO-A-85/00599 yields a product still containing relatively high amounts of the (R)-(+)-enantiomer, the undesired side-product. EP-A-239.710 further mentions that the product obtained according to the method of WO-A-85/00599 is hygroscopic and thus not stable, containing about 2% of water. One mole of crystal water would imply a water content of 5.5%. A product having a varying content of water has the drawback that the percentage of water must be analysed each time a pharmaceutical formulation shall be prepared. The above-reported Ropivacaine form should thus be characterised as an anhydrous Ropivacaine hydrochloride in unstable—and therewith undesired—form.

In addition, EP-A-239.710 teaches that the anhydrous Ropivacaine hydrochloride described in WO-A-85/00599 could not be purified any further, even not if subjected to recrystallisations from isopropanol, the solvent used therein. Although water was added, it was not possible to obtain an optically more pure, or with respect to the water content, more well defined product.

Therefore, U.S. Pat. No. 5,959,112 teaches a process for preparing the (S)-(−)-enantiomer in its monohydrate form, which is stable and which does not change by storing at ordinary room temperature and humidity. Thereto, racemic starting material pipecoloxylidide hydrochloride is liberated from its HCl salt, resolved by crystallisation with a resolving agent, and the stable crystalline product is then alkylated with a 1-halopropane. After alkylation Ropivacaine hydrochloride is precipitated by extraction with water, and obtained with a chiral purity of only about 90%. To obtain a higher chiral purity, Ropivacaine hydrochloride should afterwards be dissolved in water, whereupon hot acetone is added. The solution is then filtered as hot as possible and left for crystallisation. A yield of about 80% is reported in U.S. Pat. No. 5,959,112. However, the isolation from hot acetone is a plain method for laboratories but not easy to implement for production in the plant.

Alternatively, crystal water may be removed from the resulting monohydrate by heating at 75° C. for 16 hours, to obtain Ropivacaine in anhydrous form, according to EP-A-239.710. Such an extensive drying step makes the process hardly suitable for implementation in an industrialised process to overcome instability issues.

Hence, in the art there is a continuous need to provide cost-effective and simplified preparation routes for producing Ropivacaine hydrochloride having high chiral purity, and high yields, and in a stable form

### SUMMARY OF THE INVENTION

It is an object of the invention to provide a cost-effective process for preparing and isolating Ropivacaine base having high chiral purity.

It is also an object to provide Ropivacaine hydrochloride in stable form and having high chiral purity, suitable as an alternative to existing stable Ropivacaine hydrochloride monohydrate, preferably in a form substantially free from the corresponding R-(+)-enantiomer, containing the same amount or even less of the R-(+)-enantiomer than its monohydrate counterpart.

It is further an object to provide a simple and industrially applicable process for preparing stable Ropivacaine hydrochloride with high yield, and with the opportunity to maintain the product in stable form without requiring extensive purification steps.

It is now found that stable Ropivacaine hydrochloride in anhydrous form having a high chiral purity may be obtained starting from Ropivacaine base and preparing the hydrochloride form by using a combination of isopropanol and hydrogen chloride under water-free conditions, instead of aqueous hydrochloride and hot aqueous acetone as suggested in U.S. Pat. No. 5,959,112 and EP-A-239.710. Additional and extensive drying steps to convert Ropivacaine monohydrate into stable and anhydrous Ropivacaine are thus avoided. In view of WO-A-85/00599, WO-A-96/12700 and EP-A-1.433.782, it is considered essential in the process of the invention to start from a chirally pure Ropivacaine base.

When searching for isolation routes alternative to the aqueous isolation taught in the art, a skilled person, faced with the problem of providing anhydrous Ropivacaine in stable form, would never contemplate to incorporate isopropanol (IPA) in the preparation route, especially not in the final isolation step, starting from Ropivacaine base, as the use of isopropanol in the crystallisation of these pipecoloyl xylidide hydrochlorides has already long been associated in the art with instability (as detailed in the background to the invention).

In the present process it is important to first provide Ropivacaine base with a chiral purity of more than 95%, more preferably at least 99.5%. Lower purities would lead to crystallisation of the undesired enantiomer, and yield Ropivacaine hydrochloride in unacceptably low levels. Additional resolving steps would be required, disadvantageously introducing water—and therewith instabilities—to the system again.

Advantageously, the preparation process of the present invention results in a yield of a stable anhydrous Ropivacaine end product of typically more than 95%, preferably even more than 98%, which is far better than the yield of about 76% and 80% Ropivacaine hydrochloride monohydrate as taught in EP-A-239.710 and U.S. Pat. No. 5,959,112, respectively, without the requirement of any additional purification steps. The yield of stable Ropivacaine anhydrate is also much higher than that reported for its hygroscopic counterpart in WO-A-85/00599. The overall yield with the present process is more than 77%, compared to 53% in case of WO-A-85/00599, calculated from L-pipecolic acid 2,6-xylidide ((S)-2-Pipecoloxylidide).

“Stable” Ropivacaine anhydrate is intended to comprise Ropivacaine anhydrate which remains anhydrous over at least 2 weeks, preferably at least 1 month, without showing any significant change in water uptake, even under moisturised conditions. After a storage period of the above-defined length at a temperature of 10-50° C. and at a relative humidity of 20-80%, even 50-80%, it still exhibits a water content, as determined by standardised Karl-Fischer titration methods, of less than 2.0 wt % crystallisation water, more preferably less than 1 wt % crystallisation water. Therein, ropivacaine hydrochloride according to the present invention differs from the anhydrous form which may be obtained from its monohydrate counterpart after extensive drying (16 hours at 75° C.) as taught in EP-A-239.710. The anhydrous ropivacaine hydrochloride thus obtained still refers to the hygroscopic and not stable anhydrous form.

It is also found by the present inventors that the use of a phase transfer catalyst in the N-alkylation step further improves chiral purity, and reduces reaction time and temperature. As a reference, N-alkylation in the preparation of Ropivacaine hydrochloride in WO-A-85/00599 is reported to involve temperatures of 70° C. for about eight hours. U.S. Pat. No. 5,959,112 teaches the use of a catalyst in the alkylation step and heating, preferably to reflux temperature, because the reaction could be very time-consuming if no catalyst is used or if the reaction is performed at lower temperature. It advocates the use of a iodide catalyst, preferably sodium iodide. There is no hint towards the use of a phase transfer catalyst therein, and towards the reduction in reaction time and temperature and improved chiral purity realised therewith.

### DETAILED DESCRIPTION

The present invention thus pertains to a process for preparing stable anhydrous Ropivacaine hydrochloride of formula:

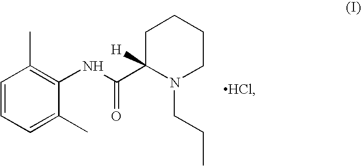

wherein the process involves adding hydrogen chloride to Ropivacaine base of formula:

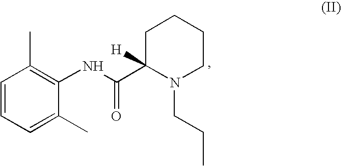

in the presence of isopropanol, wherein the process is performed under water-free conditions, and wherein the Ropivacaine base is provided having a chiral purity of more than 95%, preferably at least 97%, more preferably at least 99%, in particular at least 99.5%.

With “water-free conditions” it is understood that the mixture containing Ropivacaine hydrochloride during the preparation has a water content of less than 0.5 wt %, preferably less than 0.1 wt %, as can be determined using standardised Karl-Fischer titration methods. All solvents and starting materials used are considered water-free in the field.

More in particular, Ropivacaine base is preferably provided as dissolved in an organic solvent other than isopropanol, whereupon isopropanol and hydrogen chloride are added. The preferred organic solvent is a ketone, in particular acetone or methyl isobutyl ketone (MIBK), most preferably MIBK.

The Ropivacaine base is preferably dissolved in copious amounts of the organic solvent, typically in a concentration of 10-100 μl, more preferably 50-80 μl. During the addition the temperature is preferably maintained at 30-60° C., preferably in the range of 35-50° C. For complete conversion of base to its hydrochloride form, it is preferred to perform the addition slowly and continuously over a period from 0.5-3 hours, preferably 1-2 hours. The weight ratio of the sum of added IPA and HCl to Ropivacaine base is between 0.3:1 and 3:1, more preferably 0.5:1-2:1. If case of an organic solvent other than IPA, the weight ratio of the sum of isopropanol and hydrogen chloride to the organic solvent is preferably in the range of 1:5-1:20. Isopropanol and hydrogen chloride may be added to Ropivacaine base in a weight ratio of isopropanol:HCl of 2:1-9:1.

It the most preferred embodiment hydrogen chloride and isopropanol are added as a mixture to the Ropivacaine base dissolved in the organic solvent. The hydrogen chloride added in isopropanol is referred to as IPA.HCl. Typically, the HCl content of the IPA.HCl solution is between 10-33 wt %.

After addition, the mixture is allowed to cool down and crystallise. Afterwards, the solids can be filtered and dried according to conventional means. These steps may conveniently be performed at room temperature.

After isolation the Ropivacaine hydrochloride anhydrate thus obtained may be further processed by packaging, preferably under the same water-free conditions. It is preferred to package Ropivacaine hydrochloride anhydrate thus obtained within two weeks, more preferably within one week, most preferably within a day, with particular preference immediately after preparation.

Ropivacaine base having the high chiral purity necessary to perform the invention may be obtained by preparing it in a bi-phasic N-alkylation step, more in particular an N-propylation step, of L-pipecolic acid 2,6-xylidide, making use of a phase transfer catalyst. This is a significant improvement of the 90% chiral purity reported following conventional synthesis routes.

The mechanism may be the following: the phase transfer catalyst forms a complex with the L-pipecolic acid 2,6-xylidide in the organic phase, which complex is then transported from the organic phase to the aqueous phase containing the alkylating agent. Once formed, the N-alkylated compound transfers from the aqueous phase to the organic phase. The catalyst accelerates the reaction and makes it neat and clean.

Hence, the process further involves providing Ropivacaine base by N-propylating L-pipecolic acid 2,6-xylidide or L-pipecoloxylidide of formula

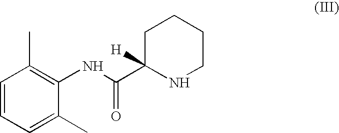

in the presence of a phase transfer catalyst (PTC), wherein the reaction involves a biphasic reaction mixture containing an alkaline aqueous phase and an organic phase.

It is known in the art to separate the undesired R-pipecolic acid 2,6-xylidide and isolate the L-pipecolic acid 2,6-xylidide of formula (III) using a resolving agent forming a stable crystallisation system with water, preferably a combination of a ketone and water. The resolution step is described in great detail in e.g. WO-A-85/00599 or U.S. Pat. No. 5,959,112. Any resolution method can be used in the process of the invention. Resolving agents that may be used are L-(−)-dibenzoyl tartaric acid or L-(−)-ditoluoyl tartaric acid.

In particular the resolution methods as published in J. Med. Chem. 14 (1971) 891-892 or Acta Chem. Scand B41 (1987) 757-761 are preferred, wherein a mixture of 2′,6′-pipecoloxylide is treated with dibenzoyl-L-tartaric acid monohydrate, whereby isopropanol is used as the preferred resolving solvent, despite of the instability of the crystallisation system that is ascribed thereto later on, see e.g. U.S. Pat. No. 5,959,112. Advantageously, the single solvent system of IPA is easy to recover and reuse, in contrast to the binary acetone-water mixture taught in the art.

L-pipecoloxylidide of formula (III) is provided to the PTC reaction form in an organic solvent, or in a mixture of organic solvents. After its preparation, it may directly be used in the N-alkylation, thus making any intermediate drying or purification steps superfluous. The preferred organic solvent is toluene.

The concentration of L-pipecoloxylidide in the biphasic reaction mixture is preferably 0.1-10 g/l, more preferably 0.5-5 μl, calculated on the total volume of the biphasic reaction mixture. The water content of the biphasic reaction mixture is typically between 20-50 wt % of the biphasic reaction mixture. An alkaline aqueous phase is understood to comprise a pH of at least 10, more preferably at least 12, most preferably even higher.

The L-pipecoloxylidide is alkylated with a 1-halopropane. The preferred alkylating reagent is n-propyl bromide or n-propyl iodide. The alkylation reaction is performed in the presence of a base. Alkylating agents and bases that can be used in the N-alkylation are appreciated by a person skilled in the art. Carbonates or hydroxides, in particular the potassium or sodium salts thereof, especially sodium hydroxide, are particularly useful as a base. The N-alkylating agent is used in an amount of 70-80 wt. % with respect to L-Pipecoloxylidide.

The phase transfer catalyst may be a quaternary ammonium or phosphonium salt, preferably a quaternary ammonium salt, more preferably a tetra-alkyl ammonium salt having C₂-C₈ alkyl, or benzyl-trialkylammonium salt, wherein the alkyl is C₂-C₈. The counter anion is preferably a halide or a hydrogen sulphate, more preferably I. In the most preferred embodiment the PTC is tetrabutylammonium iodide TBAI. For the purpose of the invention PTC is employed in catalytic amounts, preferably 10-15 wt. % of the amount of L-pipecoloxylide.

The reaction temperature during the phase transfer reaction is preferably in the range of 50-90° C., more preferably between 60-80° C. At these temperature conditions, the reaction is typically completed within 3 hours, preferably within 2 hours. Preferably, a temperature of at least 70° C. is maintained in order to complete the N-alkylation.

The invention also pertains to Ropivacaine hydrochloride in its stable anhydrous form, either packaged or unpackaged, obtainable by the process of the invention. In the context of the invention, “Ropivacaine hydrochloride anhydrate” and “anhydrous Ropivacaine hydrochloride” are considered interchangeable, meaning Ropivacaine hydrochloride containing less than 2.0 wt % crystallisation water, more preferably less than 1 wt % crystallisation water, most preferably no crystallisation water at all. The compound is further characterised in that it contains less than 0.5 area % of the corresponding R-(+)-enantiomer, a specific optical rotation [α]_{D}²⁵ of −6.4 to −6.8° (c=2 in water), and a melting interval of 260-262° C.

Analogue to the above teaching, in a further aspect the invention pertains to a process for preparing stable anhydrous (S)-(−)-1-alkyl-2′,6′-pipecoloxylidide hydrochloride, wherein alkyl is methyl, ethyl or butyl, corresponding to Mepivacaine, Lidocaine and Bupivacaine hydrochloride, and the process involves preparing them from their respective base forms, wherein the preparation involves the use of isopropanol and hydrogen chloride and is performed under water-free conditions, and to the stable anhydrous end products obtained by the water-free preparation process using isopropanol and hydrogen chloride. The Mepivacaine base, Lidocaine base and Bupivacaine base having a chiral purity of more than 95%, more preferably more than 97%, most preferably more than 99%, in particular at least 99.5% may be provided by N-alkylating L-pipecolic acid 2,6-xylidide hydrochloride in the presence of a phase transfer catalyst, wherein the reaction involves a biphasic reaction mixture containing an aqueous phase and an organic phase.

Although the principle of phase transfer catalysed N-alkylation could be extended to N-substituted alkyl groups of five or more atoms, it is known that these homologues are too toxic to function as a local anaesthetic.

The invention also pertains to pharmaceutical preparations containing the new pure anhydrous and stable compound as active ingredient: to the use of these compounds in the manufacture of pharmaceutical preparations having local anaesthetic effect. The preparation of such pharmaceutical preparations involving the new compounds falls within the ambit of the skilled person's knowledge. This also applies to the determination of the administration form and dosage of the compounds.

### EXAMPLES

### Example 1

### Preparation of Ropivacaine Base

a. Resolution

70 g (0.3 mol) 2-pipecolinoxylidide was dissolved in 350 ml IPA at RT, and filtered through a hiflow bed. The filtrate was then washed with 2×49 ml IPA and heated to 80° C. A solution of dibenzoyl-L-(−)-tartaric acid was added. This solution was prepared by dissolving 59.5 g (0.16 mol) dibenzoyl-L-(−)-tartaric acid in 170 ml IPA at 80° C. in 1 hr. The reaction was maintained at 80° C. for 1 hrs, cooled to RT and stirred for another hour. The solid was filtered and washed with 3×25 ml IPA, and dried. Dibenzoyl-2-pipecolinoxylidide-L-tartrate was thus obtained. Dry weight 58.6 g. Yield 94.6%.

b. N-Alkylation

40 g (0.048 mol) dibenzoyl-2-pipecolinoxylidide-L-tartrate, 160 ml DM water, and 240 ml toluene were mixed and stirred at RT. After heating the mixture to 60° C., 13.12 ml aqueous sodium hydroxide solution (48% w/w) was added at 60° C. in 0.5 hrs. The reaction mixture was stirred at 60° C. for another 0.5 hrs. The organic layer was separated, and the aqueous layer was discarded. 11.6 g (0.145 mol) sodium hydroxide solution (50% w/w), 3.0 g catalyst (TBAI), and 17.8 g (0.144 mol) n-propyl bromide was added at RT. The reaction mixture was heated to 70° C. and stirred for 1-2 hrs. After cooling to 60° C., 120 ml DM water was added and the mixture was stirred for 0.5 hrs. Again, the organic layer was separated, and the aqueous layer was discarded. The organic layer was then dried over sodium sulfate, filtered, concentrated and cooled to 0-5° C. The slurry was stirred for 1 hrs and filtered. After drying, Ropivacaine base was obtained. Dry weight was 21.0 g. Chiral purity ≧99.5%, Specific Optical Rotation [α]_{D}²⁵ −82.0° to −84.0° (c=2 in methanol). Yield 78.7%.

### Example 2

### Preparation of Ropivacaine Hydrochloride Anhydrate

10 g (0.03 mol) Ropivacaine base prepared according to example 1b was stirred and 130 ml methyl isobutyl ketone (MIBK) was added at RT. The mixture was heated to 40° C., 8.5 g (0.04 mol) IPA.HCl (20% w/w HCl in IPA). HCl was added at 40° C. in 0.5 hrs. The mixture was stirred for 15 min., cooled to RT and stirred for another 1 hr. The solid was filtered and dried. Dry weight 11.2 g. Chiral purity ≧99.5%. Yield 98.9%.

### Example 3

### Preparation of Ropivacaine Base

a. Resolution:

1000 g (4.3 mol) 2-pipecolinoxylidide was dissolved in 5000 ml IPA at RT, filtered through a hiflow bed, and washed with 2×700 ml IPA. The filtrate was heated up to 80° C. A solution of dibenzoyl-L-(−)-tartaric acid (850 g (2.37 mol) dibenzoyl-L-(−)-tartaric acid in 2420 ml IPA) was added at 80° C. in 2 hrs. The reaction was maintained at 80° C. for 1 hr, cooled to RT and stirred for another hour. The solid was filtered, washed with 3×350 ml IPA, and finally dried. Dry weight 859.1 g. Yield 97.0%

b. N-Alkylation

855 g (1.04 mol) dibenzoyl-2-pipecolinoxylidide-L-tartrate, 3420 ml DM water, and 5130 ml toluene were mixed and stirred at RT. The mixture was heated up to 60° C., and 280.44 ml aqueous sodium hydroxide solution (48 wt %) was added at 60° C. in 0.5 hrs. The reaction mixture was stirred at 60° C. for 0.5 hrs. The organic layer was separated and the aqueous layer discarded.

247.65 g (3.09 mol) sodium hydroxide solution (50% w/w), 64 g catalyst (TBAI), 380.7 g (3.09 mol) n-propyl bromide were added at RT. The reaction mixture was added to 70° C., stirred for 1-2 hrs, and cooled to 60° C. 2565 ml DM water was added and stirred for 0.5 hrs. The organic layer was separated and the aqueous layer discarded. The organic layer was dried over sodium sulfate, filtered, concentrated and cooled to 0-5° C. The slurry was stirred for 1 hrs, filtered and dried. Dry weight 444.6 g. Chiral purity ≧99.5%, Specific Optical Rotation [α]_{D}²⁵ −82.0° to −84.0° (c=2 in methanol). Yield 78.8%.

### Example 4

### Preparation of Ropivacaine Hydrochloride Anhydrate

419 g (1.53 mol) Ropivacaine base prepared according to example 3b was stirred and 5447 ml MIBK was added at RT. The mixture was heated up to 40° C. 355 g (1.94 mol) IPA.HCl (20% w/w) was added at 40° C. in 1.5 hrs, stirred for 15 min. and cooled to RT and stirred for another hour. The solid was filtered and dried. Dry weight 470 g. Chiral purity ≧99.5%. Yield 99%.

1-10. (canceled)

11. A process for preparing stable anhydrous Ropivacaine hydrochloride, said process involving adding hydrogen chloride to Ropivacaine base in the presence of isopropanol, wherein said process is performed under water-free conditions, and wherein Ropivacaine base is provided having a chiral purity of more than 95%.

12. The process according to claim 11, wherein Ropivacaine base is dissolved in an organic solvent other than isopropanol, whereupon isopropanol and hydrochloride are added, preferably as a mixture.

13. The process according to claim 11, wherein the weight ratio of the sum of added isopropanol and hydrogen chloride to Ropivacaine base is between 0.3 and 3.

14. The process according to claim 12, wherein said organic solvent is a ketone.

15. The process according to claim 12, wherein a mixture of isopropanol and hydrogen chloride are added to said Ropivacaine base, wherein the concentration of hydrogen chloride is 10-33 wt % of said mixture.

16. The process according to claim 11, wherein said Ropivacaine base has a chiral purity of at least 99.5%.

17. The process according to claim 11, wherein Ropivacaine base is provided by N-propylating L-pipecolic acid 2,6-xylidide hydrochloride in the presence of a phase transfer catalyst, wherein the reaction involves a biphasic reaction mixture containing an alkaline aqueous phase and an organic phase.

18. Ropivacaine hydrochloride anhydrate in its stable form, obtainable by the process according to claim 11, preferably having a chiral purity of at least 99.5%.

19. A process for preparing stable anhydrous (S)-(−)-1-alkyl-2′,6′-pipecoloxylidide hydrochloride, wherein alkyl is methyl, ethyl or butyl, and the process involves isolating it from its corresponding base, wherein said process involves the use of isopropanol and hydrochloride, and is performed under water-free conditions.

20. The process according to claim 19, wherein said base is provided by N-alkylating L-pipecolic acid 2,6-xylidide hydrochloride in the presence of a phase transfer catalyst, wherein the reaction involves a biphasic reaction mixture containing an alkaline aqueous phase and an organic phase.
